(19) United States
(12) Patent Application Publication
(10) Pub. No.: US 2011/0257560 A1
(43) Pub. Date: Oct. 20, 2011

(54) SOUND DISTORTION FEEDBACK

(76) Inventor: Kjetil Nilsen, Stavanger (NO)

(21) Appl. No.: 13/073,165

(22) Filed: Mar. 28, 2011

(30) Foreign Application Priority Data
Apr. 20, 2010 (NO) NO 20100567

(51) Int. Cl. A61B 5/103; G09B 23/32

(52) U.S. Cl. 600/587; 434/265

(57) ABSTRACT

A system is described for facilitating correct performance of CPR by providing feedback to a person performing chest compressions during basic life support or CPR training. The device is adapted to give audio signals to the user indicative of chest compressions being within a desired range or being outside of the desired range. In a first aspect the audio signal comprises two tones, a first tone that is substantially constant in pitch and a second tone that has a varying pitch. The pitch of the second tone is varying in response to a parameter of the chest compression performance. When the parameter is within a desired range, the second tone has substantially the same pitch as the first tone and when the parameter is outside the desired range, the pitch of the second tone is different from the first tone. In a second aspect the audio signal is a piece of music. This is played back at normal speed when a parameter of the chest compression performance is within a desired range and played back at a different speed when the parameter is outside off the desired range. In a third aspect the piece of music is played back with a normal range of frequencies when the parameter is within the desired range. If the parameter is outside the desired range the piece of music is played back using a cut off filter that reduces or eliminates certain frequency ranges of the piece of music.

### TECHNICAL FIELD

The present invention relates to a system for facilitating correct performance of cardio pulmonary resuscitation (CPR) by providing feedback to a person performing chest compressions during basic life support or CPR training, the device being adapted to give audio signals to the user indicative of chest compressions being within a desired range or being outside of the desired range.

### BACKGROUND ART

US 2008/0064971-A1 describes a system including a modulator that is adapted to output an audible signal having a first pitch when chest displacement within a desired range is performed. If the chest displacement is out of the desired range a signal with a second pitch will be produced. The amplitude of the signal may be pulsed to coincide with the desired frequency of the chest compression.

US 2003/0192547-A1 describes a device that can produce different types of signals to aid the rescuer, e.g., audible signals with different sound level, frequency or pitch. As an example is mentioned using a beep to indicate when chest compression is to be performed and a “breathe” when ventilation should be performed.

US 2007/0060785-A1 describes a device which is capable of giving an audible feedback in the form of a tone that is varied in frequency, e.g., the tome is ramped up in frequency during the upstroke and down in frequency during the downstroke. It is also mentioned the possibility of producing different biphasic tones to indicate when inflation should be performed and when compression should be performed.

US 2007/0049976-A1 describes a device which is capable of producing beeps with different frequencies depending on the desired chest compression rate.

Except for US 2008/0064971-A1, these prior art devices produce audible signals solely indicative of the desired chest compression frequency or depth or the desired timing of inflations and chest compressions.

US 2008/0064971-A1 provides an audible signal to the rescuer that depends on the performance. The signal is a single tone that has a first pitch when the performance is as desired and a second pitch when the performance is outside the desired range.

Although this to some degree will enable the rescuer to adjust his performance, this prior art device has some shortcomings:

The rescuer has to gain positive knowledge of which of the tones is indicating correct chest compression and which is indicating incorrect. Some people may mix up the two tones and believe that they are outside the desired range when they are within, and vice versa.

Although most people would hear the difference between the two tones, there are tone deaf persons who would have great difficulties in hearing the difference. Another disadvantage is that the user will not know how far he is from the desired range. He may lie just outside the range without knowing and may try making a significant adjustment in an attempt to get inside the range. In addition he will not know on which side of the range he is performing. An attempt to get inside of the range may result in the performance getting even further away from the desired range. The result may be that the rescuer gets alternately feedback from the two tones without knowing how he should act to get comfortably inside the range.

Consequently, there is a need for better and more intuitive feedback to the rescuer/student on how he performs compared to the desired performance.

### SUMMARY OF INVENTION

The present invention aims to provide a feedback scheme that enables the rescuer/student intuitively to know how he is performing compared to the desired performance.

In a first aspect this is achieved by a system according to the invention wherein the audio signal comprises at least two tones, a first tone that is substantially constant in pitch and a second tone that has a varying pitch, the pitch of the second tone varying in response to a parameter of the chest compression performance, so that when said parameter is within a desired range second tone has substantially the same pitch as said first tone and when said parameter is outside said desired range the pitch of the second tone is different from said first tone.

In a second aspect this is achieved by a system wherein the audio signal comprises a piece of music, said piece of music being played back at normal speed when a parameter of the chest compression performance is within a desired range and said piece of music being played back at a different speed when said parameter is outside off said desired range.

In a third aspect this is achieved by a system wherein the audio signal comprises a piece of music, said piece of music being played back with a normal range of frequencies when said parameter is within said desired range and if said parameter is outside said desired range said piece of music is played back using a cut off filter that reduces or eliminates certain frequency ranges of said piece of music.

### BRIEF DESCRIPTION OF DRAWINGS

The invention will now be described referring to the enclosed drawings, in which:

FIG. 1 shows, in a first embodiment of the present invention, a graph schematically representing a feedback based on sinus tones,

FIG. 2 shows in a second embodiment of the present invention, a graph representing two sinus tones with the same pitch and amplitude,

FIG. 3 shows in the second embodiment of the present invention, two tones with the same amplitude but with different pitch,

FIG. 4 shows, in a third embodiment of the present invention, an example of a playback of a piece of music, and

FIG. 5 shows, in a fourth embodiment of the present invention, the use of a cut-off filter.

### DETAILED DESCRIPTION OF THE INVENTION

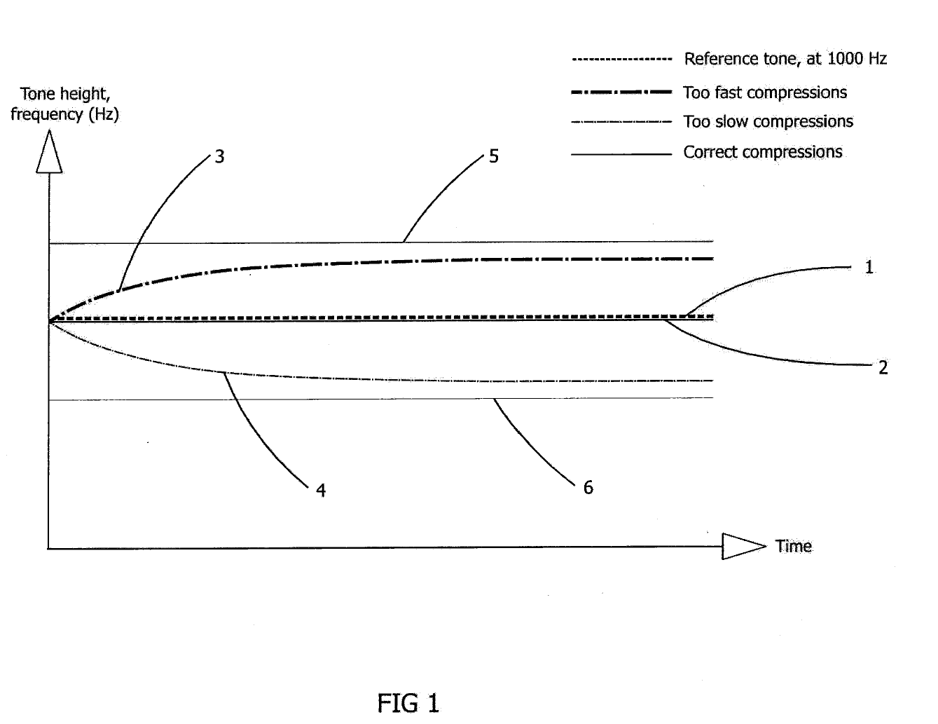

FIG. 1 shows a graph schematically representing a feedback based on sinus tones. A sinus tone is the simplest kind of noise because it consists of a single frequency, i.e. the number of occurrences of a repeating event per unit time. A sound with a frequency of 1000 Hz means that it makes the air molecules vibrate back and forth with a speed of 1000 vibrations pr. second. The human ear is trained to perceive differences in this vibration speed, and most humans can distinguish between two different sounds based on their frequency or pitch (the perceived frequency by the human ear).

However, when a tone is played alone most people will not be able to tell which frequency, or rather which tone is being played. If the tone changes most people will notice the change and can also tell if the pitch has increased or decreased. However, a relatively significant proportion of the population have problems in distinguishing between different notes. A study indicates that this condition may affect 3-6% of the population (http://brain.oxfordjournals.org/cgi/reprint/125/2/238).

The same study shows that amusical persons have great difficulties in discerning small pitch differences; the smaller the difference, the greater the difficulty. The study also shows that amusical persons, although not as good as musical persons, will perceive dissonant music as more unpleasant than consonant music. However, the study suggests that people with congenial amusia have a relatively normal ability to distinguish “sad” and “happy” music, sad meaning slow paced music in a minor key and happy being fast paced music in a major key. The same study also indicates that amusical persons can easily distinguish between familiar voices when listening to speech samples. The quality that distinguishes human voices is the frequency spectrum of which the voice is put together, i.e. the unique combination of frequencies. Hence, an amusical person will relatively easily tell the difference of a simple sound with few frequencies, and a complex sound with many frequencies.

In the simplest embodiment of the present invention the ability to distinguish dissonance from consonance is utilised. In the above mentioned study a musical piece was used for the test. However, a musical piece is by itself quite complex and may impede the ability to distinguish between consonance and dissonance.

Therefore the first embodiment of the present invention uses two tones only. A reference tone 1, which is played with a constant pitch, and a variable tone, with a pitch that can vary. The frequency of the varying tone is based on the speed of the compressions. When the rescuer compresses at the desired rate, the constant tone 1 and the varying tone 2 will have the same pitch. The two tones will be perceived as one tone only.

When the rescuer compresses faster than desired, the varying tone will be played with a higher pitch 3 than the constant tone. The faster the rescuer compresses, the higher the pitch will be.

However, the pitch of the varying tone 3 will always lie between the constant tone 1 and the closest higher harmonic 5 of that tone. Thereby the varying tone will be in dissonance with the constant tone. The rescuer will perceive these superimposed tones 1, 3 as unpleasant or at least to have a richer sound picture than the single tone 1, 2 played when the compression rate is correct, i.e. the sound will be more complex (more frequencies and disharmonies) than the correct sound.

If the compression rate is slower than the target rate, the varying tone 4 will be played with a lower frequency than the constant tone 1. The varying tone 4 will also in this case always lien between the constant tone 1 and the closest lower harmonic 6 of that tone.

The user will then be able to perceive the tone he produces as being either higher or lower than the reference tone, and adjust to the feedback by compressing faster or slower, knowing that when the produced tone is equal in pitch to the reference tone, he or she holds the correct compression pace.

It is of course also possible to let the varying tone vary with the compression depth, so that a too shallow compression depth will result in the varying tone having a lower pitch than the constant tone, and vice versa.

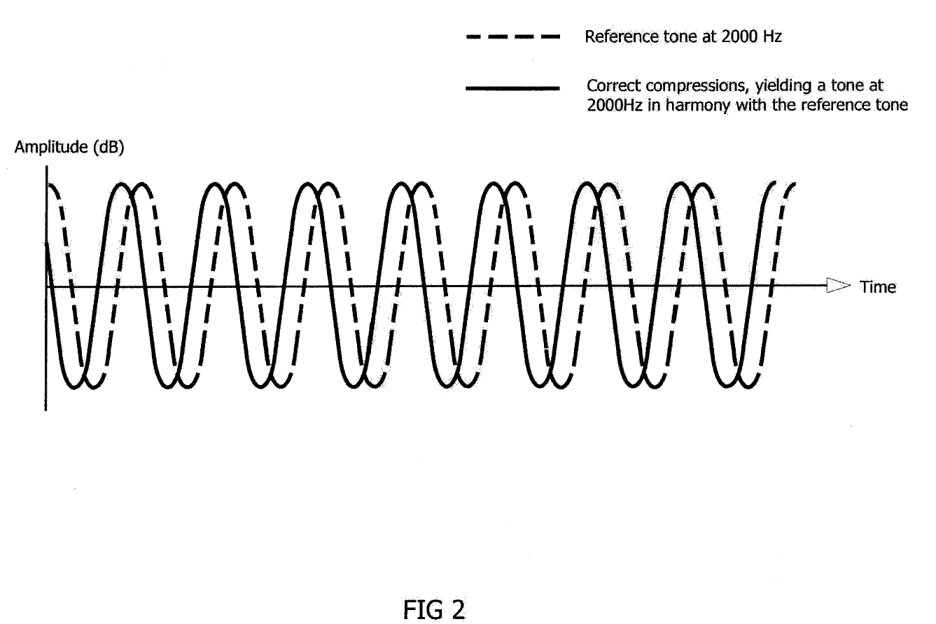

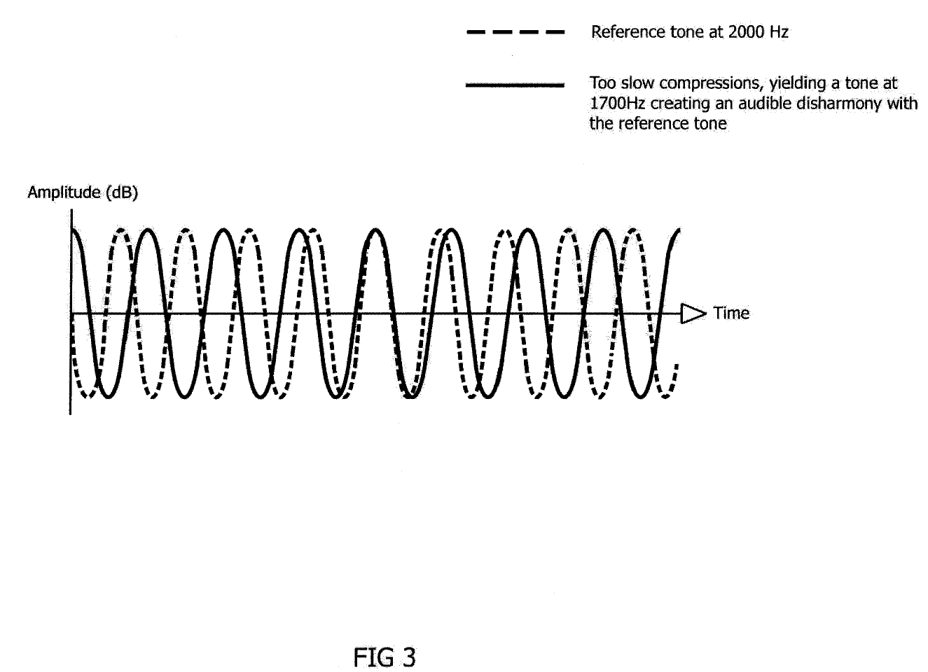

FIG. 2 shows an example of two tones 7, 8 with the same pitch and amplitude played together. The amplitude of the two tones 7, 8 will always vary in conjunction. FIG. 3 shows two tones 9, 10 with the same amplitude but different pitch. As can be seen from this figure the amplitude of the two tones 9, 10 will be out of synchronization.

In a second embodiment of the present invention a piece of music is used. In the beginning, and as long as the compression rate is correct, the music is played back at the correct speed. The musical piece can be chosen to have a beat coinciding with the desired compression rate when played back at the correct speed.

In order to indicate to the rescuer that he or she compresses with an incorrect rate, the speed of the playback can be altered. This can be done by altering the sampling rate.

A digital sound signal is commonly defined with a reference frequency, as a property of the file format, and this defines the number of sample points or recorded positions of an air molecule pr. second. This can typically be 44.1 kHz, and means that the position of the vibrating air molecule has been recorded 44100 times pr. second. The number of times the air molecule reaches its extreme positions pr second is what defines the pitch or frequency of the recorded sound. If a sound is recorded with a sample rate of 44.1 kHz, which is the standardized sampling rate of a CD, the music should also be played back using the same sample rate, 44.1 kHz, thus playing the sound with its original frequency.

However, using software techniques the playback speed can be manipulated, and it is possible to play fewer or more samples pr. second. This will then alter the pitch of the recorded sound.

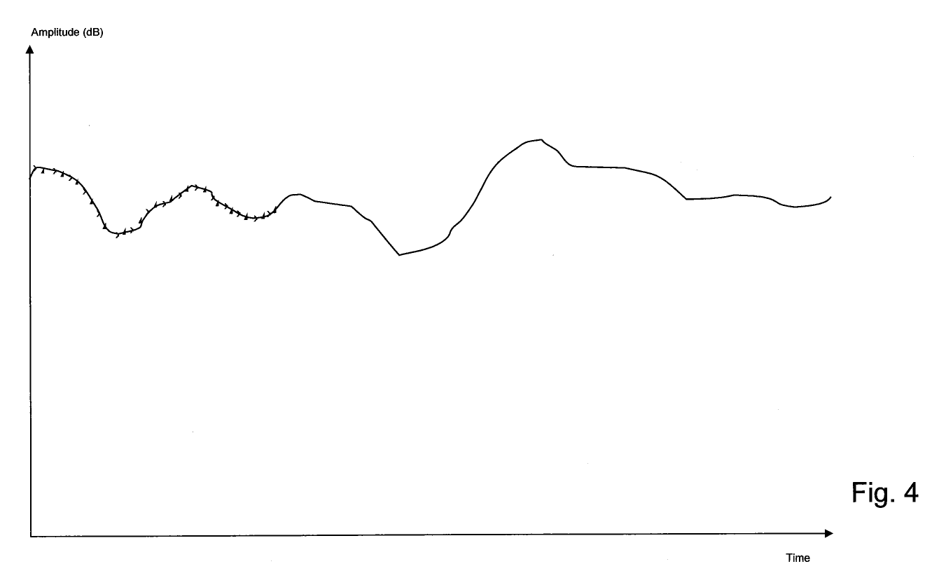

FIG. 4 shows an example of a playback of a piece of music. The x and ▴ markings on the curve 11 represents the samplings. Normal playback is done using both the x and ▴ as sampling points. If only the x points are used the sampling rate is half of the normal sampling rate. Playing the sound at this lower sampling rate will make the frequency lower, air molecules will vibrate at a slower pace, and the recorded sound will be perceived darker. At the same time it will take more time to play back the entire recording with a slower sampling rate, hence the sound will appear slower, especially if the recorded sound have a constant beat or rhythm.

The present invention utilizes this to aid the rescuer into keeping to the desired compression rate. If the compression rate of the rescuer slows down, the sampling rate is also reduced. The rescuer will hear that there is something wrong with the music and try to get the music right again. Since slower music also will sound darker, the rescuer has two characteristics of the music as an indication that something is wrong. The only thing that the rescuer can do to make the playback sound right again is to increase the sampling rate. In a similar way the music will sound too fast and light if the compression rate is too fast.

Many people lack a sense of rhythm and will have trouble keeping pace with a metronome rhythm, but will still have the ability to sense different qualitative properties of a sound, such as pitch, frequency, reverb etc. The change in sampling rate will therefore enable persons with poor rhythm sense to hold a steady compression rate within a desired range.

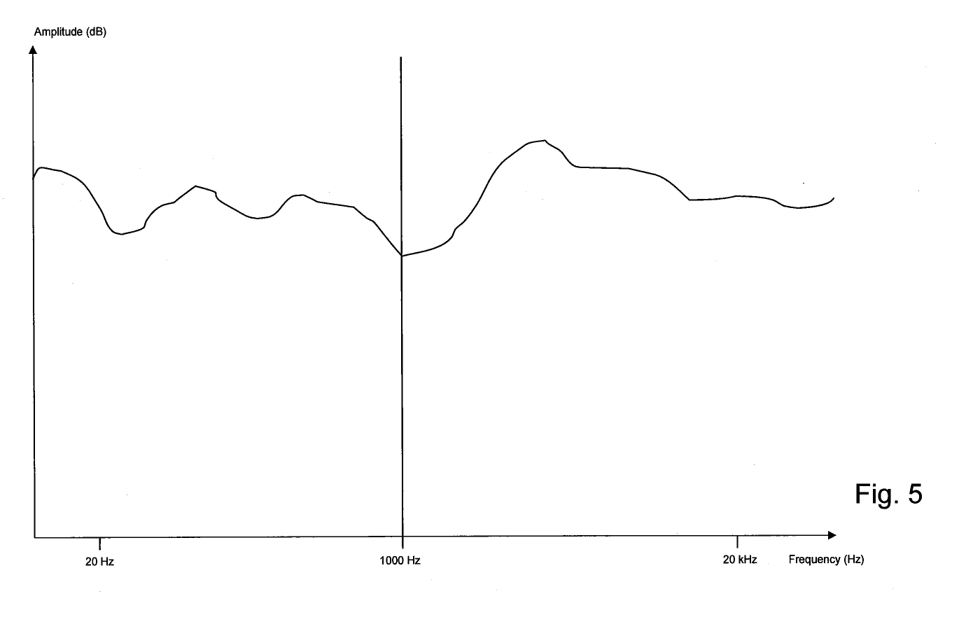

To give the rescuer a feedback that the compressions are to shallow (to deep compressions are rarely a problem), the frequency spectrum of the music may be limited in proportion to the compression depth. FIG. 5 shows an example of a frequency distribution 12 of a music piece at a given point of time. A sufficient compression depth will result in the complete frequency spectrum of the music being played back. If the compressions are too shallow, a low pass cut off filter 13 will cut off the higher frequencies of the music, so that only the frequencies below 10 kHz will be played back. The shallower the compression depth is the further down in frequency the cut off filter 13 will be set.

The result of a too shallow compression depth is that the rescuer will perceive the music as dark, woolly and poor in sound. To get the music right he will have to compress deeper.

A normal piece of music that have a fixed tempo of 100 beats per minute will have a quite notably distortion when pitched up or down by 20%.

This means that if the music is synchronized with the rate of chest compressions, having a regular playback beat at 100 compressions per minute, a rate of say 80 compressions per minute will clearly be perceived as too slow by the distorted pitch. The resulting music will sound like it's played on a cassette player with depleted batteries. Likewise a rate of 130 compressions per minute will mean that the music is pitched up 30%, a very distinguishable distortion, sounding like a cassette player that fast forwards while holding down the play button.

A normal piece of music will consist of a whole range of frequencies, typically from the low bass notes at 100 Hz up to cymbals ranging at 10.000 Hz. A low pass cut off filter 13 can for instance cut away all the frequencies above 1000 Hz. The sound is then very notably distorted.

If the music is synchronized with the depth of the chest compressions, playing back the entire frequency range for compressions above 40 mm, then compressions at, e.g., 30 mm could result in a cut off filter 13 cutting off all frequencies above 1000 Hz, as indicated in FIG. 5. This would potentially give a notion that the resulting sound is incomplete, and hence the rescuer has to press deeper to get the full musical picture.

It is also possible to use the cut-off filter as feedback on compression rate. E.g., at a compression rate less than 90 compressions per minute the filter will cut away all frequencies above a certain frequency and at a compression rate above 120 compressions per minute the filter will cut away all frequencies below a certain frequency. The rescuer will thereby perceive the music as dark when he is compressing too slow and as bright when he is compressing too fast.

The changes in pitch, sampling rate and cut off frequency can be done continuously or in increments.

In order to give the user feedback on both compression rate and depth, both the change in speed of playback and the cut off filter features of the invention can be used at the same time.

### INDUSTRIAL APPLICABILITY

The present invention is primarily intended for providing feedback on the performance of chest compression efficiency to a rescuer during CPR or a student during CPR training.

For CPR on human beings the invention may be implemented as appropriate hardware and software in a CPR feedback device, such as the one described in WO 2004/056303 or the products marketed by Laerdal Medical as CPRmeter™ and Q-CPR™.

For training purposes the invention can also be implemented in CPR training manikins. Several such manikins are marketed by Laerdal, inter alia under the trademark Resusci® Anne.

A person of skill in the audio processing field will be capable of designing the appropriate electronic circuits and software to implement the invention.

1. A system for facilitating correct performance of cardio pulmonary resuscitation (CPR) by providing feedback during basic life support or CPR training, the system comprising:
a device that gives audio signals to a user indicative of chest compressions being within a desired range or being outside of the desired range;
wherein the audio signal comprises at least two tones;
a first tone that is substantially constant in pitch;
a second tone that has a varying pitch;
the pitch of the second tone varying in response to a parameter of the chest compression performance;
wherein when the parameter is within a desired range, the second tone has substantially the same pitch as the first tone; and
wherein when the parameter is outside the desired range, the pitch of the second tone is different from the pitch of the first tone.

2. A system according to claim 1, wherein the second tone is varying between the first tone and up to but not equal to a next higher harmonic of the first tone.

3. A system according to claim 1, wherein the second tone is varying between the first tone and up to but not equal to a next higher harmonic of the first tone.

4. A system according to claim 1, wherein the first and the second tones are sinus tones.

5. A system according to claim 1, wherein the first and the second tones are pulsed with a frequency corresponding to the desired compression rate.

6. A system according to claim 1, wherein the parameter is compression depth.

7. A system for facilitating correct performance of cardio pulmonary resuscitation (CPR) by providing feedback to a person performing chest compressions during basic life support or CPR training, the system comprising:
a device that gives audio signals to a user indicative of chest compressions being within a desired range or being outside of the desired range;
wherein the audio signal comprises a piece of music;
wherein the piece of music is played back at normal speed when a parameter of the chest compression performance is within a desired range; and
wherein the piece of music is played back at a different speed when the parameter is outside the desired range.

8. A system according to claim 7, wherein the speed of the piece of music depends on a deviation of the parameter from the desired range.

9. A system according to claim 7, wherein the parameter is chest compression rate.

10. A system according to claim 7, wherein the parameter is compression depth.

11. A system according to claim 7, wherein a playback speed is adjusted by changing a sampling rate.

12. A system for facilitating correct performance of cardio pulmonary resuscitation (CPR) by providing feedback to a person performing chest compressions during basic life support or CPR training, the system comprising
a device that gives audio signals to a user indicative of chest compressions being within a desired range or being outside of the desired range;
wherein the audio signals comprise signal comprises a piece of music;
wherein the piece of music is played back with a normal range of frequencies when a parameter is within the desired range; and
wherein when the parameter is outside the desired range, the piece of music is played back using a cut off filter that reduces or eliminates certain frequency ranges of the piece of music.

13. A system according to claim 12, wherein the cut off filter is a low pass filter that eliminates frequencies above a certain frequency.

14. A system according to claim 12, wherein the cut off filter is a low pass filter that eliminates frequencies below a certain frequency.

15. A system according to claim 12, wherein the reduction or elimination of frequencies is proportional to the deviation of the parameter from the desired range.

16. A system according to claim 12, wherein the parameter is chest compression depth.
